(19) United States
(12) Patent Application Publication
(10) Pub. No.: US 2019/0088370 A1
(43) Pub. Date: Mar. 21, 2019

(54) SYSTEMS AND RELATED METHODS FOR REDUCING TRANSFERS OF PATIENTS IN NEED OF ACUTE MEDICAL CARE

(71) Applicant: Access Physicians, PLLC, Dallas, TX (US)

(72) Inventors: Kevin Houlihan, Dallas, TX (US); Eduardo Vadia, Dallas, TX (US); Christopher Michael Gallagher, Dallas, TX (US); Erica St. Angel, Dallas, TX (US); Sean McDonald, Dallas, TX (US)

(21) Appl. No.: 16/189,331

(22) Filed: Nov. 13, 2018

Related U.S. Application Data
(63) Continuation of application No. 15/599,271, filed on May 18, 2017.
(60) Provisional application No. 62/343,566, filed on May 31, 2016.

(51) Int. Cl. G16H 50/30; H04N 7/14; G16H 40/67; G06F 19/00; H04N 7/15

(52) U.S. Cl. CPC G16H 50/30; H04N 7/147; H04N 7/15; G06F 19/00; G16H 40/67

(57) ABSTRACT

Disclosed herein are unique systems and methods for remotely located physician specialists to diagnose and treat patients in a non-acute care facility having symptoms typically indicating a need for transfer to an acute care facility. The disclosed principles minimize the transfer of patients from a non-acute care facility to an acute care facility when not needed. The disclosed principles provide for the use of telemedicine units by geographically remote physician specialists to diagnose and treat patients in a non-acute care facility. If the physician specialist determines via the telemedicine unit that transfer of the patient to higher level of facility is needed, then the patient is transferred. If the physician specialist determines that the patient's new, elevated condition may be treated in the current, lower level facility, the physician specialist can prescribe a treatment for the current facility's staff to implement, even under the supervision of the physician specialist if it is needed. This unique approach is possible by providing an advanced telemedicine unit for use by the physician specialist that is geographically remote from the patient and his current lower level of care facility. Accordingly, the disclosed telemedicine program uses remote physicians having a higher level of skill or specialty to diagnose and treat patients currently located in a lower level healthcare facility than that type of specialty physician is typically found.

### RELATED APPLICATIONS

This disclosure is a continuation of U.S. patent application Ser. No. 15/599,271, filed May 18, 2017, which claims the benefit of U.S. Provisional Application No. 62/343,566, filed May 31, 2016, both of which are herein incorporated by reference in their entirety for all purposes.

### TECHNICAL FIELD

The present disclosure relates to the diagnosis and treatment of patients in healthcare facilities, and in particular to systems and methods for remotely located physician specialists to diagnose and treat patients in a non-acute care facility having symptoms typically indicating a need for transfer to an acute care facility.

### BACKGROUND

Obtaining adequate medical care in all locations within the U.S. is an ongoing challenge. For example, only about 10 percent of physicians practice in rural America despite the fact that it contains nearly one-quarter of the U.S. population. Making matters worth, according to some studies, rural Americans also have greater health and socioeconomic challenges. Specifically, demographic information shows that rural Americans tend to be of lower income, have more chronic conditions, rely more on food stamps and other social services, are less likely to have employer-sponsored insurance, and are less likely to have prescription drug coverage, when compared with urban dwelling Americans.

According to some numbers, large states like Texas have rural hospitals that provide access to routine and emergency health care for only about 15 percent of the state's population; however, these rural hospitals cover 85 percent of the state's geography, according to the Texas Organization of Rural and Community Hospitals. As a result, there are areas in Texas that are more than 100 miles away from the nearest hospital. Other states with large rural geographies have similar coverage issues. For example, about three-fourths of the 2,050 rural counties in the U.S. include a primary care health professional shortage area. Nearly one in 10 rural counties has no primary care physician at all.

This healthcare shortage is even more acute for specialists. Among the same studies discussed above, statistics have shown that rural areas have about 40 specialists per 100,000 residents, compared with 134 specialists per 100,000 residents in urban areas. Also, the more highly specialized the physician may be, the less likely he or she will settle or practice in a rural area.

Additionally, death and serious injury accidents account for about 60 percent of total rural accidents, as compared to only about 48 percent in urban areas. One reason for this disparity is that in rural areas, prolonged delays can occur between a motor vehicle accident, the call for emergency medical services (EMS), and the arrival of EMS personnel. Many of these delays are related to increased travel distances and personnel distribution across the rural response area. National average response times from motor vehicle accident to EMS arrival in rural areas is about 18 minutes, as compared to about 10 minutes in urban areas.

One answer to the shortage of physicians in rural areas has been telemedicine, which has spread rapidly within the last decade. The number of patients cared for through variations of telemedicine technology has risen to about 10 million people in rural areas, as well as urban settings, according to the American Telemedicine Association. Typically, such conventional telemedicine approaches involve the use of what amounts to a video conferencing system that is mobile, and thus may be moved into a patient's room where a physician may see the patient using the equipment, and converse with both the patient and any healthcare provider, such as a nurse, that is present with the patient.

However, these conventional approaches to telemedicine are limited to what can be seen through a video camera, or what is told to the remotely located physician by the patient or a healthcare practitioner. Moreover, conventional uses of telemedicine has thus far only been for the purpose of “providing” a physician to a geographically distant patient for the purpose of diagnosing and treating a patient remotely. Accordingly, what is needed in the art is a unique telemedicine system and related uses that do not suffer from the deficiencies of conventional equipment for, and approaches to, telemedicine. The disclosed principles provide such a unique solution.

### SUMMARY

To overcome the deficiencies of the conventional approaches to telemedicine, the disclosed principles provide unique equipment and techniques for employing such equipment in a unique manner. The disclosed principles allow a patient's elevated condition to be diagnosed and often treated while the patient remains in their current level of healthcare facility. Accordingly, the disclosed principles minimize the transfer of patients from, e.g., a long-term care facility to a short-term acute care facility. To accomplish this, the disclosed principles provide for the use of telemedicine units by geographically remote physician specialists to diagnose and treat patients in a lower level of care facility than the physician specialist would otherwise be employed. If that physician specialist determines that transfer of the patient to higher level of facility is needed, then the patient is transferred. If the physician specialist determines that the patient's new, elevated condition may be treated in the current, lower level facility, the physician specialist can prescribe a treatment for the current facility's staff to implement, even under the supervision of the physician specialist if it is needed. This unique approach is possible by providing an advanced telemedicine unit for use by the physician specialist that is remote from the patient and his current facility. Accordingly, the disclosed telemedicine program uses remote physicians having a higher level of skill or specialty to diagnose and treat patients currently located in a lower level healthcare facility than that physician is typically found.

Numerous embodiments and advantages associated with each such embodiment are discussed in further detail below. In one embodiment, a method for reducing Return To Acute Care transfers (RTAs) of patients in a non-acute care facility may comprise determining if a patient currently provided for in a non-acute care facility is suffering one or more newly arising symptoms that may require acute care. The method would then include deploying a Telemedicine Acute Care Unit for the patient, while the patient remains in the non-acute care facility, if the patient is determined to be suffering such new symptoms. The method then includes diagnosing the patient's newly arising symptoms by a physician specialist, geographically remote from the non-acute care facility, using the Telemedicine Acute Care Unit, wherein the physician specialist controls functions of the Telemedicine Acute Care Unit to diagnose the patient and communicate with the patient and non-physician specialist personnel located with the patient. In this embodiment, if the remote physician specialist determines that the diagnosis of the patient requires transfer to an acute care facility, the method comprises instructing such transfer via the Telemedicine Acute Care Unit. However, if the remote physician specialist determines that the diagnosis of the patient does not require transfer to an acute care facility, the method comprises instructing a treatment plan for the patient via the Telemedicine Acute Care Unit and for implementation by non-physician specialist personnel at the non-acute care facility.

### BRIEF DESCRIPTION OF THE DRAWINGS

The detailed description that follows, by way of non-limiting examples of embodiments, makes reference to the noted drawings in which reference numerals represent the same parts throughout the several views of the drawings, and in which:

FIG. 1 illustrates a continuum of healthcare needs of patients proportional to the healthcare capabilities of a healthcare facility;

FIGS. 2A and 2B collectively illustrate a comparison of the conventional healthcare treatment model and the unique conceptual approach of the disclosed principles, when telemedicine is employed to each approach;

FIG. 3 illustrates one exemplary embodiment of an advanced telemedicine unit in accordance with the disclosed principles;

FIG. 4 illustrates one embodiment of a Telemedicine Acute Care Unit implemented by a remotely located physician to diagnose a patient remotely in accordance with the disclosed principles;

FIG. 5A illustrates a flow diagram setting forth one embodiment of a method of reducing Return To Acute transfers (RTAs) of patients in a healthcare facility in accordance with the disclosed principles;

FIG. 5B illustrates a flow diagram setting forth another embodiment of a method of reducing RTAs of patients in a healthcare facility in accordance with the disclosed principles;

FIG. 6 illustrates a flow diagram continuing the process flow of the embodiments of reducing Return To Acute transfers RTAs of patients in a healthcare facility discussed with reference to FIG. 5A or 5B;

FIG. 7A illustrates a graph of actual RTA transfer rates of all of the patients in four distinct long-term non-acute care facilities to an acute care facility both before and after implementing a telemedicine program in accordance with the disclosed principles; and

FIG. 7B illustrates the reduction in RTA transfer rate for the first four months of one facility illustrated in FIG. 7A after a telemedicine system and process in accordance with the disclosed principles was implemented.

### DETAILED DESCRIPTION

In view of the foregoing, through one or more various aspects, embodiments and/or specific features or sub-components, the present disclosure is thus intended to bring out one or more of the advantages that will be evident from the description. The present disclosure makes reference to one or more specific embodiments by way of illustration and example. It is understood, therefore, that the terminology, examples, drawings and embodiments are illustrative and are not intended to limit the scope of the disclosure.

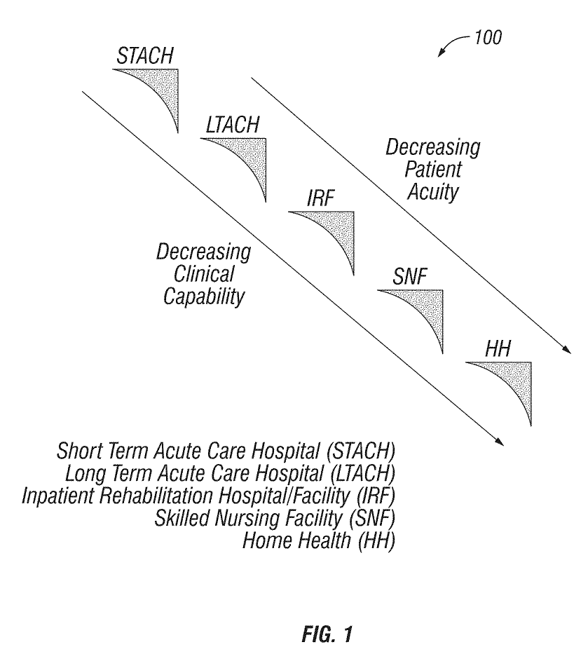

FIG. 1 illustrates a continuum 100 of healthcare needs of patients proportional to the healthcare capabilities of a healthcare facility. The continuum 100 illustrates the linear relationship between patient needs and facilities in today's world. At the top of the spectrum is Short-Term Acute Care Hospitals (STACH), which are those facilities capable of handling the most severe acute needs of patients. Commensurate with these facilities are the needs of patients with short-term acute care needs. These needs could include diagnoses any acute life-threatening situations, from heart attacks and strokes to gunshot wounds. At the bottom of the spectrum is the Home Health (HH) facility which is likewise commensurate with patient conditions that are mild enough that patients may be treated at home using, for example, a home nurse. Accordingly, the continuum 100 illustrates, at the top, the most immediate needs of a patient which requires treatment by some of the most specialized healthcare professionals and equipment, and, at the bottom, the least immediate needs of a patient requiring treatment by either no healthcare professional (in the case patient self-treatment) or some of the least specialized healthcare professionals (in the case of a home nurse or simply an assistant).

As the continuum 100 moves from the most severe to the least, Long-Term Acute Care Hospitals (LTACH) are next down from the STACH. These facilities still require specialized healthcare professional, often the same as those found in STACHs, but where the patients' needs are less immediate, although still considered life-threatening if left untreated. Next is the Inpatient Rehabilitation Hospital/Facility (IRF), which typically includes those facilities that are equipped and staffed to treat the long-term needs of patients that do not have acute care (i.e., life-threatening,) requirements. These encompass hospital facilities where patients suffering from non-life-threatening conditions still need to remain in a facility for monitoring and treatment. Next down on the continuum 100 is the Skilled Nursing Facilities (SNF) which includes those facilities equipped and staffed to handle the long-term care of patients who do not necessarily require the needs of a normal hospital. Thus, SNFs may only be staffed with nurses and other non-physician personnel, or perhaps only a non-specialized staff physician. Each of the LTACHs, IRFs and SNFs can encompass the “long-term care” discussed in this disclosure.

In additional to the directly proportional relationship of facility capability and staff with the severity of patient needs illustrated by the continuum 100, the average cost of treatment in terms of both facility and physician expense (based on specialty capabilities of both) follows this same relationship. Accordingly, the most costly facility stays and physician (and perhaps other healthcare staff) are in the STACHs, while the least cost is associated with the HH scenario. Consequently, in order to keep a patient's healthcare costs as low as possible, the goal must be to keep the patient in the lowest facility on the continuum 100 possible, while still providing the patient sufficient treatment for their condition(s). In the current healthcare landscape, the patient is kept in the level of facility having the equipment and staff sufficient to treat the patient's condition(s); however, the disclosed principle move away from that model in manner that still provides the patient the level of treatment they require, but in less costly level of facility. This unique deviation from the conventional healthcare model is discussed in further detail below.

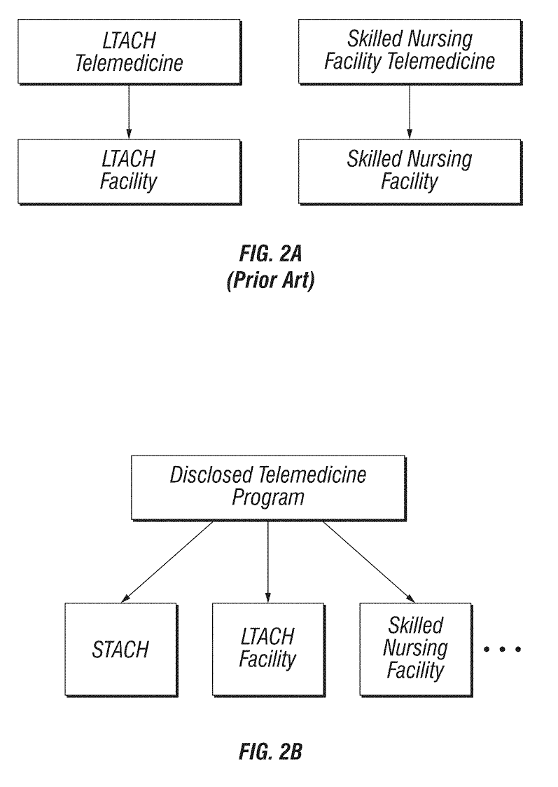

FIGS. 2A and 2B collectively illustrate a comparison of the conventional healthcare treatment model and the unique conceptual approach of the disclosed principles, when telemedicine is employed to each approach. FIG. 2A sets forth a block diagram of the conventional healthcare treatment model employing telemedicine for use by physicians to treat patients. Specifically, in the conventional approach a Long-Term Acute Care Hospital (LTACH) telemedicine unit is employed to diagnose and treat patients within a corresponding LTACH facility. Similarly, FIG. 2A illustrates a Skilled Nursing Facility telemedicine unit employed to diagnose and treat patients in a Skilled Nursing Facility. Accordingly, in the conventional to telemedicine, the telemedicine unit is simply used to replace the physical presence of the physician or other healthcare professional that would otherwise be present at the facility in which the patient is located. Stated another way, conventional telemedicine is used to reduce or eliminate the need for a physician typically located at the level of facility he/she is skilled to diagnose and treat to be physically present in that facility. Such an approach is often successful in reducing the costs associated with keeping one or more physicians of that skill level in that facility, and as such is simply used to substitute the physically present physician of a certain level (commensurate with the facility they practice within) with a virtual physician of the same skill level/occupation. Consequently, a physician skilled for working in, for example, a long-term acute care facility is simply replaced with a virtual physician of the same skill level for that facility.

FIG. 2B illustrates a conceptual block diagram of a healthcare treatment model employing telemedicine in accordance with the disclosed principles. In the disclosed approach, a single telemedicine program is employed for multiple levels of healthcare facilities. More specifically, in the illustrated example, a single telemedicine program is implemented for each of a Short-Term Acute Care Hospital (STACH) facility, a Long-Term Acute Care Hospital (LTACH) facility, and a Skilled Nursing Facility. To accomplish this, the disclosed principles, which are discussed in further detail below, employ a telemedicine unit for use by a healthcare professional having a skill level/specialty higher than the level of care of the facility the patient is currently located.

The conventional approach to telemedicine can address the patient's current needs for the level of facility he or she is located by using remote physician having skills commensurate with that level of care. However, when such patient suffers a new condition, or deterioration of his current condition, such that the level of care now required by the patient exceeds that of the local or remote physician associated with that facility, the conventional approach is to transfer that patient to a facility that has physicians skilled in the higher level of care (i.e., specialty) now required by the patient. In the case of a patient that is currently in a long-term care facility that suffers a new condition (or deterioration) that may require short-term acute care, the conventional approach has the patient immediately transferred to the wing or new facility that has the physicians trained to diagnose and treat the new/deteriorated condition. This is called a Return To Acute care transfer (RTA).

As should be expected, the costs associated with such RTAs can be high, both for the transport of the patient (e.g., from one facility to another), but also the costs of now having the patient stay in the short-term acute care facility so that they may be diagnosed. In those cases where the patient's new condition justifies staying in the new, more expensive short-term acute care facility, then the costs of the new facility is also justified. But in those cases where the new condition of the patient did not result in the need for the patient to be transferred, the costs associated with the patient's stay, even if temporary, in the short-term acute care facility did not need to be incurred. Instead, the patient's change in condition could have been diagnosed while still in the original long-term care facility, and any new treatment done in that same facility without transferring the patient. However, in the conventional approach, such a facility would not typically have a physician specialized to diagnose and treat the patient's elevated condition in those cases where the patient's elevated condition exceed the expertise of the long-term care facility staff. This is because the conventional healthcare model places physicians and other staff of a given skill level or expertise only within the facility associated with that same level of specialty. Consequently, there would not be a physician with the advanced specialty in that long-term care facility to provide a diagnosis and treatment of the patient's elevated condition; hence, the patient's transfer to a properly staffed acute care facility.

In contrast, the approach of the disclosed principles allows the patient's elevated condition to be diagnosed and often treated while the patient remains in their current level of healthcare facility. Accordingly, the disclosed principles minimize the transfer of patients from, e.g., a long-term care facility to a short-term acute care facility. To accomplish this, the disclosed principles provide for the use of telemedicine units by physician specialists to diagnose and treat patients in a lower level of care facility than the physician specialist would otherwise be employed. If that physician specialist determines that transfer of the patient to higher level of facility is needed, then the patient is transferred. If the physician specialist that the patient's new, elevated condition may be treated in the current, lower level facility, the physician specialist can prescribe a treatment for the current facility's staff to implement, even under the supervision of the physician specialist if it is needed. This unique approach is possible by providing an advanced telemedicine unit for use by the physician specialist that is remote from the patient and his current facility. Accordingly, as shown in FIG. 2B, the disclosed telemedicine program uses remote physicians having a higher level of skill or specialty to diagnose and treat patients currently located in a lower level healthcare facility than that physician is typically found.

The situations giving rise to the need for a telemedicine program in accordance with the disclosed principles go beyond the mere costs associated with RTAs. For example, in many geographic locations, access to a higher level physician than what facility may be available is limited or even nonexistent. In those situations, patients suffering from conditions above the level of care of their available facility must either transport themselves to another location, if even possible for many third-world patients, or suffer the ultimate consequence of their geographically limited options. With a telemedicine program as disclosed herein, the services of a higher level physician may thus be employed in such patients' more limited (in terms of healthcare services/capabilities) facilities. In these situations, it is not the use of telemedicine to provide increased access to physicians commensurate with the geographically limited facility, it is the providing of a higher level of skill physician where one, even with the presence of conventional telemedicine, is simply not provided. Moreover, even where physicians of higher skill level are available to visit a lower level facility, not only may their numbers be so limited that they cannot properly service all such facilities, but the time needed for such physician specialists to reach those patients in lower level facilities may be too long for some patient's conditions to tolerate. Also, in many cases the lower level facilities cannot afford to staff a full time higher level physician specialist, and thus a single physician specialist can accommodate multiple lower level facilities. In this respect, lower level facilities are only required to “purchase the part of the physician specialist they use.” In sum, efficiency of treatment by such physicians of higher skill level in lower level facilities is also provided by the telemedicine of the disclosed principles. Not only can patients in lower level facilities obtain higher level physician care without the cost and time of transfer, but that care can be obtained at a far quicker rate. And even from the physician specialists' point of view, these physician specialists can now expand their practices from their usual higher level of care facility to lower level facilities, thereby increasing physician income simultaneously.

In addition to these situations in which a telemedicine program in accordance with the disclosed principles may be employed, throughout all scenarios there are a number of advantages achieved by this unique type of program. As discussed above, the RTAs of patients in a non-acute care facility is significantly reduced by those situations in which the remote physician specialist employs the program to diagnose and establish a treatment plan for those patients that do not need to be transferred to acute care. Accordingly, since as discussed above the costs associated with the facility and healthcare staff (especially physicians) substantially increases as the level of care increases, but reducing the RTA transfer rates, patient costs are also reduced by avoiding those higher cost facilities. These reductions in cost are not only for patient or patient insurance funds, but also for those patients employing Medicare or other social program to pay for their care. This reduces overall costs to those social programs in the long term, which can thereby reduce overall costs to tax payers. Similarly with insurance companies, where savings to insurance companies over time would reduce overall costs to those companies, thereby reducing premiums or at the least limiting premium increases. Furthermore, the financial stability of lower level facilities is greater since when a patient is temporarily transferred to short term acute care for diagnosis and treatment, those facilities lose revenue they would have otherwise collected on those days the patient is in acute care.

Advantages extend beyond mere dollars and cents too, such as reducing the time needed for patients in need of higher level of care to receive that higher level of care. That could be due to the time needed for a physician specialist to physical arrive at the lower level facility where the patient is located, or due to the time involved with transferring a patient that is suddenly suffering from acute systems. These reductions in diagnosis and treatment time directly translate into saving patient lives. Even the reduction in transfers times allows that time to be used for actual treatment, which again translates into saving lives. Furthermore, by reducing the number of RTAs, the bed occupancy rates in the high level facility is also reduced, which allows more beds to be open for those patients that must be transferred to the higher level facility. Yet another advantage is that by “flipping” the conventional engagement model using the disclosed telemedicine program (i.e., the physicians contact the nursing staff), the disclosed principles have shown to increase early detection situations. In this manner, physician specialists employing the disclosed system have been able to identify elevated condition situations at a much earlier stage the in the conventional approach, be it conventional telemedicine or face-to-face. By such earlier identification of an elevating condition, the disclosed techniques provide for treatment at an earlier stage, often with a much lower impact solution. Thus, the care by the physician specialist for patients in a lower level facility becomes less reactive and the patient often ends up experiencing fewer acute situations. Still further, the telemedicine program of the disclosed principles improves quality of life for both patient and their family as they have more stability and are able to stay at a single facility in many cases. Not only is this a matter of convenience, but it may further mean either gets to remain closer to home and family, and thus less travel.

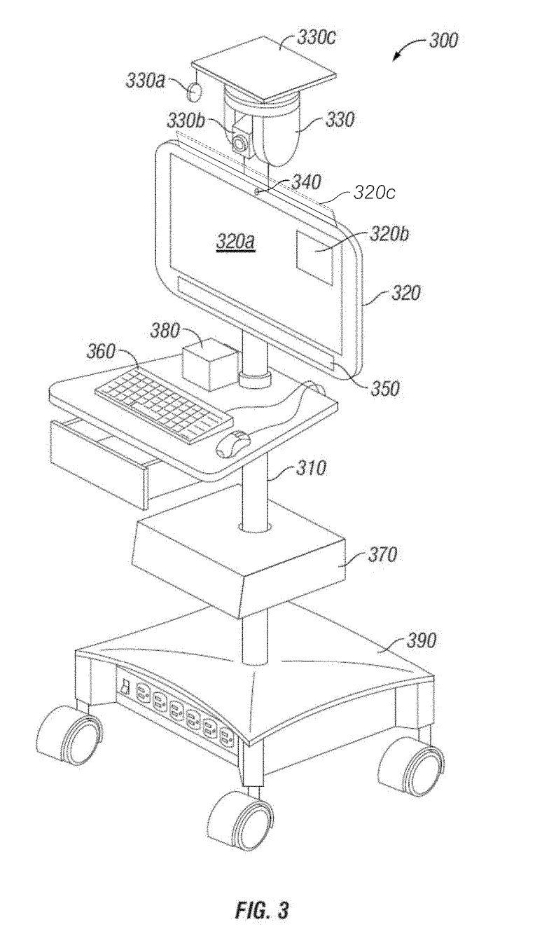

Turning now to FIG. 3, illustrated is one exemplary embodiment of an advanced Telemedicine Acute Care Unit 300 in accordance with the disclosed principles. The Telemedicine Acute Care Unit 300 may be employed for the diagnosis and treatment of a patient by a physician that is geographically remote from that patient. For example, the physician and patient may be separated by thousands of miles or could be separated by one or more floors of the same medical building.

In this embodiment, the Telemedicine Acute Care Unit 300 includes a support structure 310 configured to secure all of the components of the Telemedicine Acute Care Unit 300 in a single moveable unit. All surfaces of the Unit 300 may be constructed having an antimicrobial coating to combat infection by inhibiting the growth of bacteria and mildew, as well as permitting repeated wiping down of the Unit 300 with antimicrobial solutions. In exemplary embodiments, the mast of the support structure 310 may be configured as hollow so that cabling may be housed within the mast so as to not only remove it from view, but to keep such cabling from interfering with users or other equipment, or even simply from becoming snagged as the Unit 300 is moved. The mast may also be constructed so as to provide shielding to any cabling on the Unit 300, for example, to prevent interference during use of equipment or components of the Unit 300. Furthermore, although the mast of the support structure 310 is illustrated as having a round cross-section, it should be understood that the mast may also have a square, rectangular, triangular, or any other shaped cross-section, as desired.

The support structure 310 may also include a sturdy handle, for example, secured to and around a portion or all of the mast, at a comfortable height to permit grasping by a user maneuvering the Unit 300. Such a handle may even be sized such that some or all of the handle laterally extends farther out than components, base and/or shelves of the Unit 300, and thus protects those components or shelves from being bumped as the Unit 300 is maneuvered around. Among the components secured in the Telemedicine Acute Care Unit 300 by the support structure 310 is a display unit 320. The display unit 320 may be used to present the image of a remotely located physician (320a) that is employing the Telemedicine Acute Care Unit 300 to diagnose and treat a patient. In advantageous embodiments, the display unit 320 is selected so that the remote physician being displayed is life-size, which would provide human-scale features and eye contact (e.g., their head is displayed as normal human size) to patients viewing the remote physician on the display unit 320. The Unit 300 is configured to be accessible by such remote physicians through any type of conferencing hardware and/or software, such as through a desktop, mobile application, internet browser, or a dedicated similar video communications unit.

Also, the display unit 320 may be used to display information (320b) to the patient or a healthcare provider located with the patient. For example, such information may include textual information on an ailment the remote physician believes the patient is suffering from, or perhaps treatment options for the patient's diagnosed condition. Also, the display unit 320 may be used to display images or video pertaining to the patient's diagnosed condition, or again showing how treatment options for the patient may help. Of course, any information useful for the diagnosis and treatment of a patient may be shown on the display unit 320.

The Telemedicine Acute Care Unit 300 also includes a moveable camera unit 330 held in place by the support structure 310. To better assist the remote physician diagnosing and/or treating the patient, the camera unit 330 may be controllable by the remote physician. As such, the physician may pan and tilt the camera unit 330, as well as zoom in or out with the lens 330a of the camera unit, in order to provide him- or herself a desirable view of the patient during their diagnosis or treatment. In advantageous embodiments, the camera unit 330 includes optical zoom capabilities, as opposed to digital zoom technology, so as to permit a remote physician zoom capabilities with little to no loss of resolution. Focus controls may also be received from the remote physician's equipment so that the physician has ultimate image control. Such a camera 330 may also include a locking mechanism to secure its position during movement of the Unit 300, for example, to prevent its shaking and losing position should the Unit 300 need to be moved during its use. Such a locking mechanism may be motion-activated such that it operates to secure the camera 330 in position automatically if the Unit 300 is moved, and then unlocks when the Unit 300 comes to rest again. Alternatively or in addition, such the camera 330 may include a stabilization mechanism, such as a Steadicam® mechanism, so as to maintain camera 330 stability should the Unit 300 be moved during its use. A microphone 340 is also included in the Telemedicine Acute Care Unit 300 to capture audio from the patient's side, such as information provided by the patient or other healthcare provider located with the patient, and transmitted to the remote physician specialist. Also, the Telemedicine Acute Care Unit 300 includes audio speakers 350 to provide audio from the remotely located physician to patient's side the during their diagnosis or treatment of the patient. In the illustrated embodiment, the speakers 350 are provided as part of the display unit 320, but in other embodiments the speakers 350 may be separate from the display unit 320.

In addition to the primary communication devices discussed above, the Unit 300 may also include backup conferencing unit. The backup conferencing unit may be mounted on the support structure 310 of the Unit 300, but on the side opposite to the display unit 320. By mounting the backup conferencing unit in this location, it is kept out of the way of the other conferencing components on the Unit 300 during their use. However, should one or more of the primary conferencing components, such as the display unit 320, the camera unit 330, or the microphone 340, the Unit 300 may simply be rotated around 180 degrees in order to have the backup conferencing unit facing the patient. The backup conferencing unit would be configured with video capture and display capabilities, such as a video display screen for displaying video and images captured from the remote physician specialist's equipment, and a camera for capturing video of the patient, local workers or medical equipment with the patient. In addition, the backup conferencing unit would be configured to have audio capture and broadcast capabilities, such as a microphone for capturing audio from the patient's location, and one or more audio speakers for broadcasting audio provided from the remote physician specialist's location. In some specific embodiments, the backup conferencing unit may be a tablet computer having these capabilities, such as an Apple iPad® or a Microsoft Surface Pro® tablet computer. Of course, a proprietary conferencing unit may also be created for use as the backup conferencing unit.

Although the backup conferencing unit may not be as fully versatile as the primary conferencing components of the Unit 300, it would be configured to provide at least a minimum level of video conferencing capabilities so that the remote physician specialist can continue their diagnosis and treatment of the patient should one or more of the primary conferencing components fail during use of the cart. This redundant backup capability of the Unit 300 is an invaluable component of the Unit 300 since use of the Unit 300 would typically occur, in accordance with the disclosed principles, when the patient is experiencing one or more symptoms indicative of a life-threatening condition, and thus require immediate attention by a physician. In such cases, should a primary conferencing component of the Unit fail during this time of immediate need, the worker near the Unit 300 need only turn the Unit 300 around and turn on the backup conferencing unit so that the diagnosis and treatment of the patient's new symptoms can continue. Without such a backup conferencing unit to engage in such emergency situations, the patient suffering the new life-threatening symptoms would need to be immediately transferred to an acute-care facility, as in the conventional manner discussed above, or be diagnosed by a local healthcare worker in the long-term care facility that is likely not physician specialist. Neither of these alternatives is desirable as the first approach requiring transfer of the patient is not only time consuming, but may also turn out to have been unnecessary, and thus costly, had the physician specialist been able to finish their diagnosis. The second approach leaves the patient at great risk since their symptoms may not be diagnosed correctly by the local long-term care facility staff, due to their lack of specialty training.

The illustrated embodiment of the Telemedicine Acute Care Unit 300 also includes peripheral input devices 360 for manually providing information to either a local or network storage system. For example, the input devices 360 may include a keyboard and mouse, as illustrated, but may also include other types of devices such as an integrated trackpad instead of a mouse, as well as removable USB peripherals or storage devices. Information input using such devices 360 could be for storing data locally in a hard drive device, or such information may be transmitted using a communications unit 370 (with wireless communications antenna 330c) of the Telemedicine Acute Care Unit 300. Also, the peripheral input devices 360, along with the communications unit 370, may be connected to the backup conferencing unit, either directly or indirectly. This allows any input devices 360 to also be used with the backup conferencing unit should the need arise, and allows the backup conferencing unit to communicate fully using the communications unit 370. The communications unit 370 may be wireless or hard wired into a local area network of the facility where the patient is located. Such connections would allow information regarding the patient to be input into their medical records via the Telemedicine Acute Care Unit 300, or simply to provide information to another area of the facility or even outside the facility. Also, the Unit 300 may include components or a platform configured to monitor its own performance, and which may include automated alerts when components or communications of the Unit 300 have been compromised or is not functioning at peak performance.

In advantageous embodiments, the Unit 300 may first be connected during use using the communications unit 370 and using a Wi-Fi or other local area wireless network via its antenna 330c. In addition, such Units 300 may include a wired network connections, such as an Ethernet connection, in case the wireless network where the Unit 300 is located is not functioning. Also, in some embodiments, the Unit 300 may also include cellular network capabilities. Such embodiments are advantageous in situations where the facility's entire network is down, and thus the Unit 300 may still be employed using the mobile communications data network. Having the capability to obtain a communications connection in all three manners provides a triple redundancy for the Unit's 300 connectivity, which is especially important when the Unit 300 is employed in delicate medical procedures. Moreover, all network communications by the Unit 300 may be configured to have a minimum of the HIPAA-compliant 256-bit AES encryption standard for video conferencing.

Furthermore, the communications unit 370 or other connectivity equipment of the Unit 300 may include an override capability for either or both of the wired and wireless networks to which it can connect. More specifically, any communications or computer network is subject to bandwidth issues, for example, based on the number of devices connection to such network or the amount of bandwidth any connected device is taking. As the Unit 300 disclosed herein is employed during medical evaluations and procedures, consistent connectivity is of the upmost importance. Accordingly, the connectivity equipment of the Unit 300 can be configured to take priority of network resources while in used, or while in use for certain procedures. In this respect, the connectivity equipment can either be connected with a default priority over other connected devices/equipment on the same network, or it may be configured to connect normally and then have the capability to take priority over other connected devices/equipment, even to the extent of bumping devices/equipment from a network if needed. Moreover, if multiple Units 300 are connected to the same network, such prioritization capability of the connectivity equipment of each Unit 300 may be configured to determine priority based on what procedure is being performed with each Unit 300. For example, if a first Unit 300 is being used in delicate medical procedure while a second Unit 300 is being used for observing a patient or medical equipment of the patient, the need to maintain connectivity for the first Unit 300 can be determined to take priority over the second Unit 300, and thus usurp bandwidth or overall connectivity as discussed above. In such embodiment, the multiple Units 300 may be interconnected to determine such priority as each Unit 300 is activated for a particular use, or the Units 300 may include a manual control that permits a remote physician or a local user of a Unit 300 to choose such prioritization during use of the Unit 300.

The Telemedicine Acute Care Unit 300, in accordance with the disclosed principles, also includes a telemetry input unit 380. Specifically, the telemetry input unit 380 is configured to receive information from equipment or tools physically located with the patient in the lower level facility. Typically, the telemetry tools connected to the telemetry unit 380 are tactile-based medical tools. Some examples of such tactile-based medical tools could include a stethoscope, otoscope, ophthalmoscope, or any other medical tools that could be employed in contact with the patient by the physician specialist if he or she were present with the patient. In other embodiments, the tactile-based telemetry tools may be configured to transfer actual tactile sensations from someone touching the patient to a remote physician specialist wearing equipment, such as telemetry receiving gloves, which mimic the tactile sensations experienced by the person actually contacting the patient so that the remote physician specialist can experience the tactile sensations of touching the patient as if they were present with patient. Accordingly, such various types of telemetry tools and equipment can change depending on the specialty of the remote physician specialist. Monitoring equipment located with the patient at the lower level facility may also be connected to the telemetry input unit 380 to provide telemetry information to a remote physician specialist, and each connection with either tools or equipment may be through a wired or wireless connection to the unit 380. Such monitoring equipment may be tactile-based so as to have associated components physically contacting a patient, or they may comprise remote sensing technology.

Moreover, the Unit 300 includes a power supply 390 which may be wired via a standard electrical outlet, or may be have a battery therein to allow operation of the Unit 300 even in times of power outages. Advantageously, the power supply 390 includes both wired power capability and a battery-powered back-up (e.g., kept charged by the wired power connection) to ensure no power loss when using the Unit 300, or at a minimum a significant battery back-up time, such as one hour or more, to permit finishing of the ongoing medical diagnosis or procedure thereby reducing risk and inconvenience to the patient and physician. The power supply 390 in this embodiment is comprised in a base of the Unit 300, which itself includes wheels for providing mobility of movement to the Unit 300. Moreover, although four wheels are illustrated on this embodiment of the Unit 300, alternative embodiments may include five or six wheels for added stability, or even a greater number of wheels, if desired. Also, wheel locks may be included on one or more of the wheels on the base, allowing for the Unit 300 remain in a specific location and position during its use. Such securing allows a physician specialist or other user to ensure they do not lose visibility of the patient or equipment in the room during use of the Unit 300, which typically occurs if the Unit 300 is bumped by personnel in the same room, or if is pulled by any cables connected to the Unit such as any medical devices connected via the telemetry input unit 380.

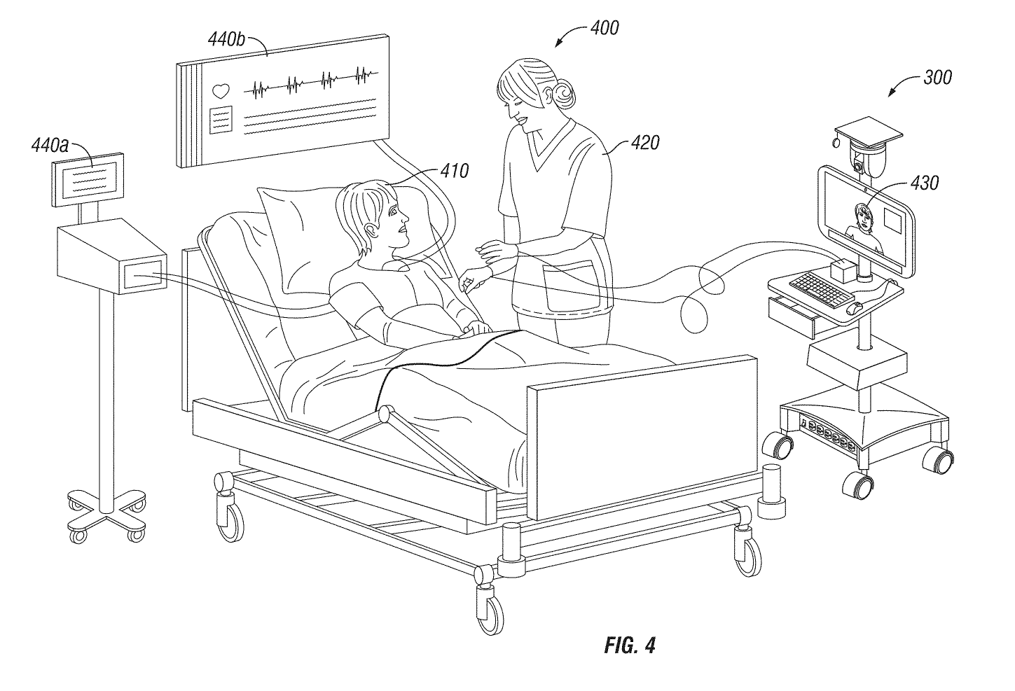

Looking now at FIG. 4, illustrated is a scenario 400 where a Telemedicine Acute Care Unit, such as the Unit 300 discussed with reference to FIG. 3, is implemented by a remotely located physician specialist to remotely diagnose a patient in a lower level care facility. In this embodiment, the patient 410 is staying at a long-term healthcare facility, and thus requires monitoring and treatment by personnel with the level of skill for that facility. For example, a healthcare professional 420 with a skill level commensurate with the level of the facility is able to diagnose and treat the patient 410.

When the patient 410 begins to experience symptoms that suggest a condition that exceed the capabilities of the facility and its personnel, the Unit 300 may be immediately brought it by the local healthcare professional 420. Using the Unit 300, a physician specialist 430, who has more specialized training than the healthcare capabilities of the facility and personnel of the facility, can diagnose the patient's current or new condition. To do so, the physician specialist 430 employs the Unit 300 to either visually inspect the patient 410 and the equipment 440a, 440b monitoring the patient 410, or employ the telemetric capabilities of the Unit 300 to connect to the equipment 440a, 440b to obtain patient information. To enable a geographically remote medical specialist, especially during emergency conditions, to deliver high quality of medical care to a patient, from the standpoint of close inspection and diagnosis, and to ensure that local medical personnel, such as nursing personnel, are enabled to concentrate on patient treatment, the remote physician instructs rather than expends time and effort manually positioning a video camera or a mobile emergency center unit having a video camera, or even passing patient information to the remote physician. Thus, it is desirable that the remote medical practitioner have the capability of independently causing the video camera to move as desired for efficient visual inspection of the patient, including close-up viewing of selective portions of the anatomy of the patient as well as any patient monitoring equipment connected to the patient.

Additionally, in the illustrated embodiment the Unit 300 is equipment with tactile feedback devices, such as tactile gloves 450. These unique gloves 450 may be worn by the healthcare professional 420 to physically contact the patient 410. The physician specialist 430 may wear corresponding tactile feedback gloves (not illustrated) which are configured to recreate the tactile sensations felt by the healthcare professional 420 wearing gloves 450. With this technology, the physician specialist 430 can simply direct the healthcare professional 420 to contact specific areas of the patient 410. Of course, other telemetric tactile feedback devices may also be employed with a Unit 300 in accordance with the disclosed principles.

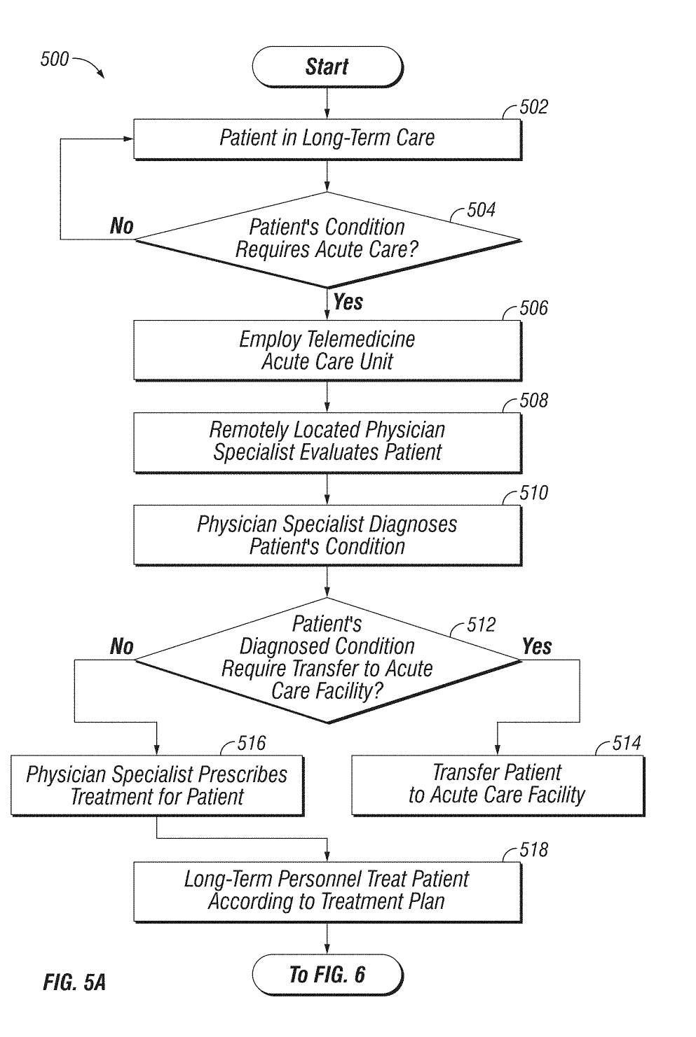

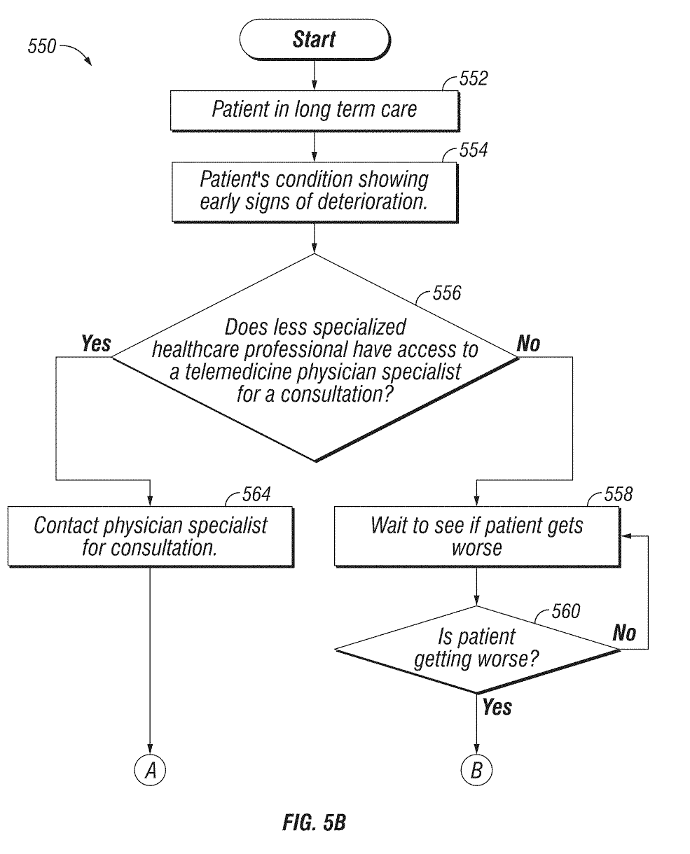

Turning now to FIG. 5A, illustrated is a flow diagram 500 setting forth one embodiment of a method of reducing Return To Acute transfers (RTAs) of patients in a healthcare facility, in accordance with the disclosed principles. Within fully capable healthcare facilities, such as hospitals, there are typically both short-term acute care areas and long-term care areas. As used here with reference to the flow diagrams, “acute care” refers to a branch of healthcare services where a patient receives active but short-term treatment for a severe injury or episode of illness, an urgent medical condition requiring emergency treatment, or during recovery from surgery. And as used herein “long-term care” is a branch of healthcare services for patients requiring an extended period of care in a medical facility, such as for an extended period of recovery from or treatment for an injury, illness or surgery. Accordingly, in medical terms, acute care for health conditions is opposite from long-term care.

Another distinct difference between acute care and long-term care is the cost involved, as discussed in detail above. In this respect, the costs associated with short-term acute care is greater than the costs associated with long-term care. Such cost differential is based on the various differences in the facilities and personnel associated with acute care versus long-term care. Acute care typically requires a greater amount of various medical equipment so that the facility is prepared for any number of emergency or otherwise critic healthcare needs. Also, due to the critical nature of the needs of patients' in acute care facilities, physician specialists are required in order to immediately diagnose and treat the critical needs of acute care patients. In contrast, long-term care typically requires only general medical equipment used in the everyday care of patients, and perhaps some specific equipment directed to some specific needs of the patients staying in the long-term facility. Additionally, contrary to acute care facilities, the daily care for patients in long-term care facilities is typically possible by less specialized healthcare professionals, such as nurses or general practice physicians. These differences allow long-term care facilities to operate far less expensively, which in turn translates to less expense to their patients and/or their insurance providers. Accordingly, it is generally more cost effective to maintain patients in long-term care facilities rather than have them in short-term acute care facilities.

With this information in mind, the disclosed principles provide techniques to reduce the costs to both the facilities and patient or their insurance providers for those patients in long-term care facilities who suddenly need acute care. In conventional practice, when a patient in a long-term care facility suddenly requires acute medical care, the patient is immediately—and quickly—moved from the long-term care facility to the acute care facility for such treatment. Once the patient arrives in the acute care facility, specialty equipment needed to diagnose/treat the patient, or perhaps to keep the patient alive, is needed. Additionally, one or more specialty physicians are also needed to assess the patient and provide a diagnosis and treatment for the patient's determined condition. Moreover, all of this has occurred within the acute care facility, thus not only occupying valuable bed space in the acute care ward, but also incurring the acute care costs associated with the bed space and personnel.

Instead of continuing with the conventional practice outlined above for patients in long-term care facilities who suddenly need acute care, the disclosed principles provide for a new and unique technique of handling these patients. By having more specialized physicians do more proactive investigation and thus catching acute situations earlier, the disclosed principles reduce the “sudden-ness” of the need for acute care and a lower acuity of care that is needed. Looking at the flow diagram of FIG. 5A, the exemplary process under the disclosed principles begins at a Step 502 where a patient is currently provided for in a long-term care facility in the conventional manner.

At a Step 504, it is determined whether the patient who is currently under long-term care has had an episode that that would require acute care. For example, the patient stable in long-term care may suddenly experience symptoms related to a heart attack. If the decision at Step 504 is No, such if the symptoms go away relatively quickly, then the process simply returns to Step 502 where the patient continues to undergo long-term care in the facility in the same manner as before or for any new ailment that only requires treatment by a long-term care facility. To make this determination, typically a nurse or similar level of healthcare professional located with the patient asks a physician in that facility for their assistance with they believe that symptoms are occurring that could require acute care. This is wrought with problems and often results in the patient's need not being identified quickly enough such that critical situations can be prevented. Thus, even if the local physician is available quickly, or is even the healthcare professional seeing the patient's symptoms change or arise, then time for contacting a physician specialist is often too long for the patient's needs. However, with the disclosed telemedicine program, the physician specialist can be available as quickly as possible. Thus, if at Step 504 the decision is Yes and the patient has suffered something that may require acute care, and thus the long-term care facility is not equipped to diagnose and treat the acute care ailment, the process moves to Step 506.

It should be noted that in conventional practice, the patient who is now suffering something that requires acute care is transferred to an acute care facility for diagnosis and treatment. However, in accordance with the disclosed principles, at Step 506 the exemplary process would have the personnel at the long-term care facility employ a Telemedicine Acute Care Unit, such as the Unit 300 discussed above with reference to FIG. 3, while the patient remains in the long-term care facility. To employ the Telemedicine Acute Care Unit, any personnel in the long-term care facility need only roll the Unit into the patient's room and locate it in a position where the camera unit can view the patient, as well as where a long-term healthcare worker can operate the Acute Care Unit so as to assist the remote physician specialist treat the patient. Alternatively, and perhaps more importantly, the telemedicine program of the disclosed principles also includes “patient presenting” capabilities where the patient can initiate the consultation with the remote physician specialist. Also, the remote physician specialist may also initiate use of the telemedicine unit, for example, if the remote physician specialist is already monitoring the patient's vital signs remotely.

Once the Acute Care Unit is employed, at Step 508 in the exemplary process, a remotely located physician specialist employs the Acute Care Unit to evaluate the patient's condition. The remotely located physician in this exemplary disclosed process is specially trained in diagnosing the patient's newly arising condition that may require acute care, as well as treating that condition when it is determined that acute care is in fact required by that condition, while any local physicians with the patient are not so trained. Thus, as used herein, the “remote physician” or “remote medical practitioner” is an academically trained specialist in this regard, as compared to any local medical personnel, whether physicians or not. When a remotely located patient is being treated, especially during emergency treatment at a remotely located emergency facility, the patient's condition may not be well known. It is thus desirable for a medical specialist practitioner, located at a remote facility, to have the capability of controlling the orientation of a patient inspection video, including panning up or down, right or left and actuating a zoom feature of the video lens. This feature will permit the medical practitioner remote from the emergency situation to conduct independent patient inspection and to discuss aspects of the patient's condition with the local medical personnel, and perhaps also with the patient, during the time the local medical personnel are engaged in the conduct of independent patient care of treatment activities at the direction of the remote medial practitioner.

To conduct the evaluation, the remote physician specialist employs several features of the Acute Care Unit. For example, the remote physician employs the camera on Unit to see not only the patient, but any of the number of patient monitoring equipment that is connected to and monitoring the patient, just as if they were in the room with the patient. Also, the Unit may be configured to communicate with any of the patient monitoring equipment so that the information provided by such equipment is transmitted to the remote physician, rather than the remote physician reading the monitoring equipment displays via the camera unit. Also, the Unit may be configured with telemetry devices, such as those discussed above with reference to FIG. 4. Furthermore, the remote physician may talk to the patient and/or any of the long-term care personnel with the patient to continue to gather information. The remote physician may also instruct the long-term care personnel to conduct one or more tests on the patient in order to assist the remote physician in this or her diagnosis.

Once all of the pertinent the information is gathered by the remote physician using the features available on the Acute Care Unit, the remotely located physician specialist can provide a diagnosis of the patient at Step 510. At decision Step 512, it is determined if the remote physician's diagnosis of the patient requires the patient to be transferred to the Acute Care Facility. If the decision of the remote physician specialist is that the patient should be so transferred, at Step 514 the patient is immediately transferred to the Acute Care Facility for treatment of their condition. In exemplary embodiments, certain “codes” may cause the order to transfer the patient to an Acute Care Facility. Examples of such acceptable diagnoses by the remote physician specialist could be hemodynamically unstable gastro intestinal bleeding; specific types of heart attacks; a hemodynamically unstable pulmonary embolism; conditions requiring urgent surgical intervention (perforated appendix, gallbladder, etc.; an acute neurologic event that requires an MRI or neurologist evaluation; and static epilepticus (continued seizures). Of course, these are only examples of some diagnoses done by the remote physician specialist that could still require transferring of the patient to an acute care facility.

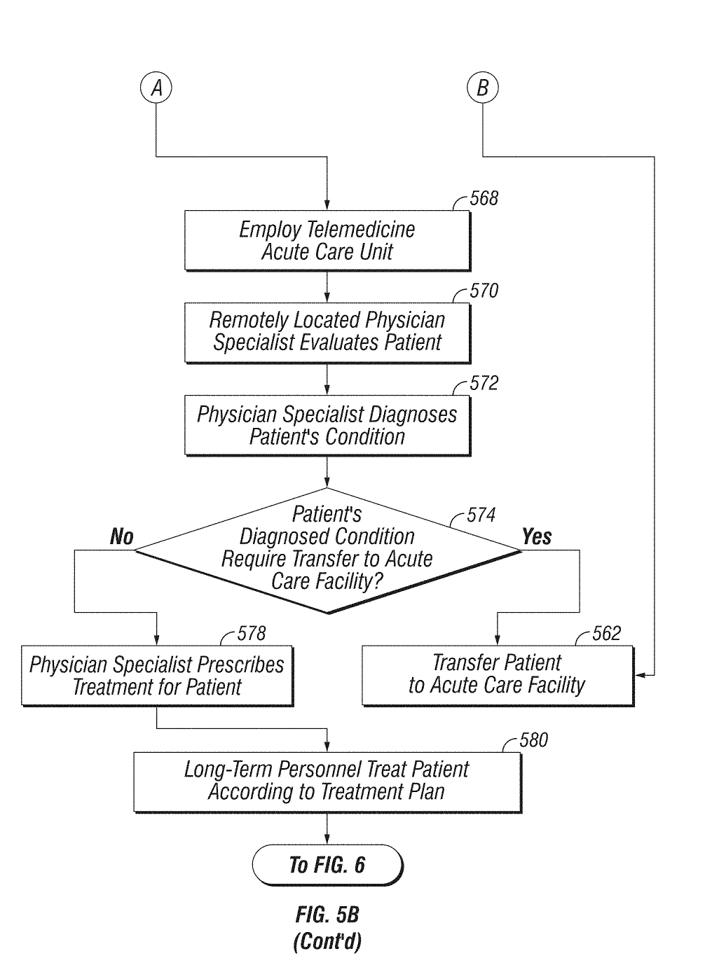

However, if at Step 512 the prognosis of the remote physician is that the patient does not need to be transferred to the Acute Care Facility, at Step 516 the remote physician creates a treatment plan for the patient in need of acute care. Specifically, the remote physician can again employ various features of the Acute Care Unit to communicate the treatment plan. For example, the remote physician can dictate the treatment plan to long-term care personnel with the patient. Also, the remote physician may enter information into the computer network at his or her location, and then that treatment information can be centrally located for access by the long-term care personnel, or it may be transmitted to the long-term care facility as needed. In some embodiments, to speed up the treatment process even further, the remote physician may be able to issue treatment plan information to various areas of the long-term care facility simultaneously, such as the facility's internal pharmacy and staff in various departments, thus permitting each department to receive their department's portion of the treatment plan as quickly as possible. Once the remote physician specialist has issued the treatment plan for the patient's acute care condition, at Step 518 the long-term personnel located with the patient implement the remote physician specialist's treatment plan. After this embodiment of a process in accordance with the disclosed principles ends, the process moves onto to the process of FIG. 6.

Looking now at FIG. 5B, illustrated is another flow diagram 550 setting forth another embodiment of a method of reducing Return To Acute transfers (RTAs) of patients in a healthcare facility, in accordance with the disclosed principles. This exemplary process under the disclosed principles begins at a Step 552 where a patient is currently provided for in a long-term care facility in the conventional manner. At Step 554, the patient is beginning to show signs of deterioration in their condition.

At a Step 556, it is determined whether the less specialized healthcare professional with the patient at the lower level facility has access to a specialized (i.e., higher, specialty training) physician for consultation on the patient's deterioration. If the answer is No, then the healthcare professional with the patient simply waits to see if the patient's condition further deteriorates. The process would then move to Step 560 where it is determined if the patient is in fact worsening. If the patient's condition is no longer worsening, the process moves back to Step 558 to wait to see if the condition does begin to worsen. If, however, at Step 560 it is determined that the patient's condition continues to deteriorate, the process moves to Step 562 where the patient is transferred to an acute care facility. The process would then stop.

If at Step 556, if it is determined that the less specialized healthcare professional with the patient at the lower level facility does have access to a specialized physician for consultation on the patient's deterioration, the process moves to Step 564, where the physician specialist is consulted. For the consultation, the process moves to Step 568 where the personnel at the long-term care facility employs a Telemedicine Acute Care Unit, such as the Unit 300 discussed above with reference to FIG. 3, while the patient remains in the long-term care facility. Once the Acute Care Unit is employed, at Step 570, the remotely located physician specialist employs the Acute Care Unit to evaluate the patient's condition, as discussed in detail above.

Once all of the pertinent the information is gathered by the remote physician using the features available on the Acute Care Unit, the remotely located physician specialist can provide a diagnosis of the patient at Step 572. At decision Step 574, it is determined if the remote physician's diagnosis of the patient requires the patient to be transferred to the Acute Care Facility. If the decision of the remote physician specialist is that the patient should be so transferred, at Step 562 the patient is immediately transferred to the Acute Care Facility for treatment of their condition. However, if at Step 574 the prognosis of the remote physician is that the patient does not need to be transferred to the Acute Care Facility, at Step 578 the remote physician creates a treatment plan for the patient in need of acute care. Specifically, the remote physician can again employ various features of the Acute Care Unit to communicate the treatment plan. Once again the remote physician can dictate the treatment plan to long-term care personnel located with the patient, or the remote physician may enter information into the computer network at his or her location, and then that treatment information can be centrally located for access by the long-term care personnel, or it may be transmitted to the long-term care facility as needed. Once the remote physician specialist has issued the treatment plan for the patient's acute care condition, at Step 580 the long-term personnel located with the patient implement the remote physician specialist's treatment plan. After this alternative embodiment of a process in accordance with the disclosed principles ends, the process moves onto to the process of FIG. 6.

Turning now to FIG. 6, illustrated is another flow diagram 600 constituting a continuation of the process flows of either FIG. 5A or 5B. As the exemplary process flow in accordance with the disclosed principles moves from FIG. 5A or 5B to FIG. 6, the long-term care personnel are implementing the treatment plan established by the remote physician specialist for the patient's diagnosed condition that would typically require treatment in an acute care facility.

At decision Step 610, it is determined if the treatment plan established by the remote physician specialist is successful for the patient's condition. If the determination is that the treatment plan so instituted by the long-term care personnel has been successful, and thus the patient's condition no longer is such that treatment in an acute care facility would be warranted under conventional practice, the process moves to the Step 620, where the long-term care personnel continues their long-term care of the patient as was occurring before the patient suffered new symptoms that required the attention of the remote physician specialist. If the determination is that the treatment plan of the physician specialist instituted by the long-term care personnel has not been successful, and thus the patient's condition is still such that treatment in an acute care facility would be warranted under conventional practice, the process moves to the Step 630, where the Telemedicine Acute Care Unit is again deployed for use by a remotely located physician specialist. The remote physician specialist need not necessarily be the same remotely located physician that diagnosed the patient initially and thereafter prescribed a treatment plan, but in some embodiments it may well be the same physician. In either situation, at Step 640 the remote physician specialist employs the Acute Care Unit such that the patient's condition is again evaluated. As before, any of the Unit's features may be employed by the remote physician specialist to conduct his or her evaluation of the patient's condition.

At decision Step 650, it is determined whether the patient's current condition is such that the patient should be transferred to an acute care facility. It such situations it may be that the treatment provide by the long-term care personnel was not sufficient, or it may be that additional treatment available only in an acute care facility may be needed for the patient. If the remote physician specialist determines that the patient should be transferred to an acute care facility, the process moves to Step 660 where the patient is immediately transferred for treatment at the acute care facility, and after which the process ends. However, if it is determined at Step 650 that the patient need not be transferred to an acute care facility, the process moves to Step 670 where the treatment of the patient will continue in the long-term care facility. More specifically, the remote physician specialist may determine that the original treatment plan prescribed for the patient simply needs to continue to be provided by the long-term personnel. Alternatively, the remote physician specialist may provide a different diagnosis for the patient's condition, and thereafter prescribe a new treatment plan for that diagnosed condition. Also, the remote physician specialist may confirm that the prior diagnosis of the patient's condition was correct in their opinion, but thereafter prescribes a new treatment plan for that diagnosed condition.

As the treatment plan of the patient provided by the remote physician specialist is implemented by the long-term care facility, the process moves to decision Step 680. At Step 680, it is determined if the patient has recovered from the condition that would have typically led to the patient being transferred to an acute care facility. In this embodiment of the disclosed process, if it is determined at Step 680 that the patient has not recovered or perhaps improved with the continued treatment by the long-term care personnel, the process moves to Step 660 where the patient is immediately transferred to the acute care facility. In accordance with the disclosed principles, this determination may also be made by a remote physician specialist again employing the Telemedicine Acute Care Unit to evaluate the patient's condition. Alternatively, the determination may be made by the long-term care personnel implementing the remote physician specialist's treatment plan on the patient. However, if it is determined at Step 680 that the patient's acute care condition has improved or been alleviated, then the process moves to Step 620 where the long-term care personnel continue their previous long-term care of the patient. Thereafter, the process ends.

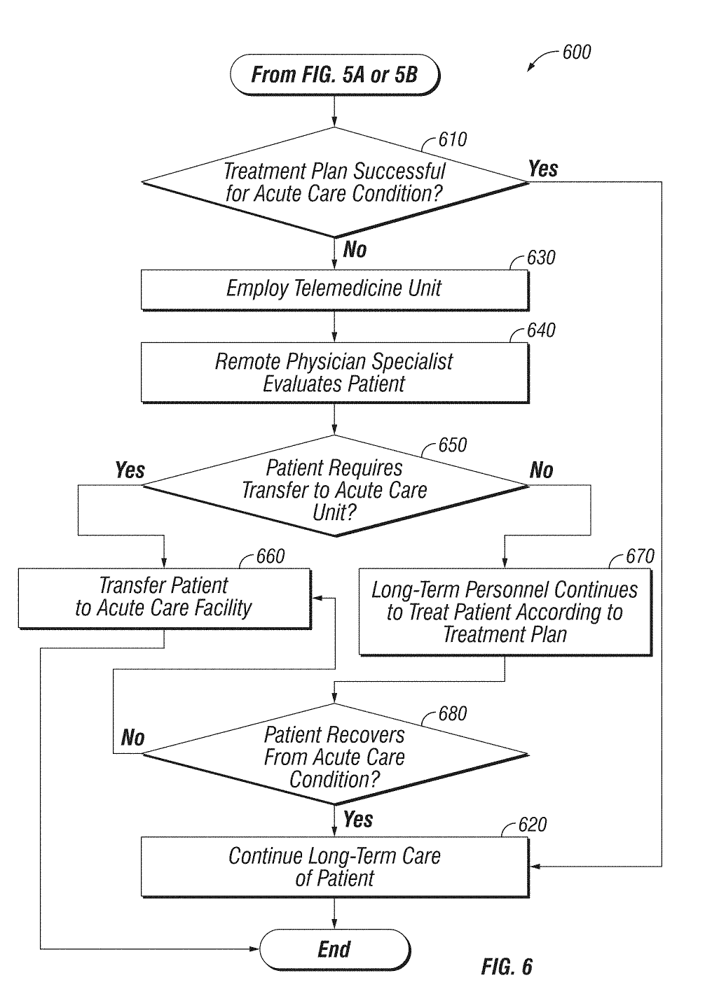

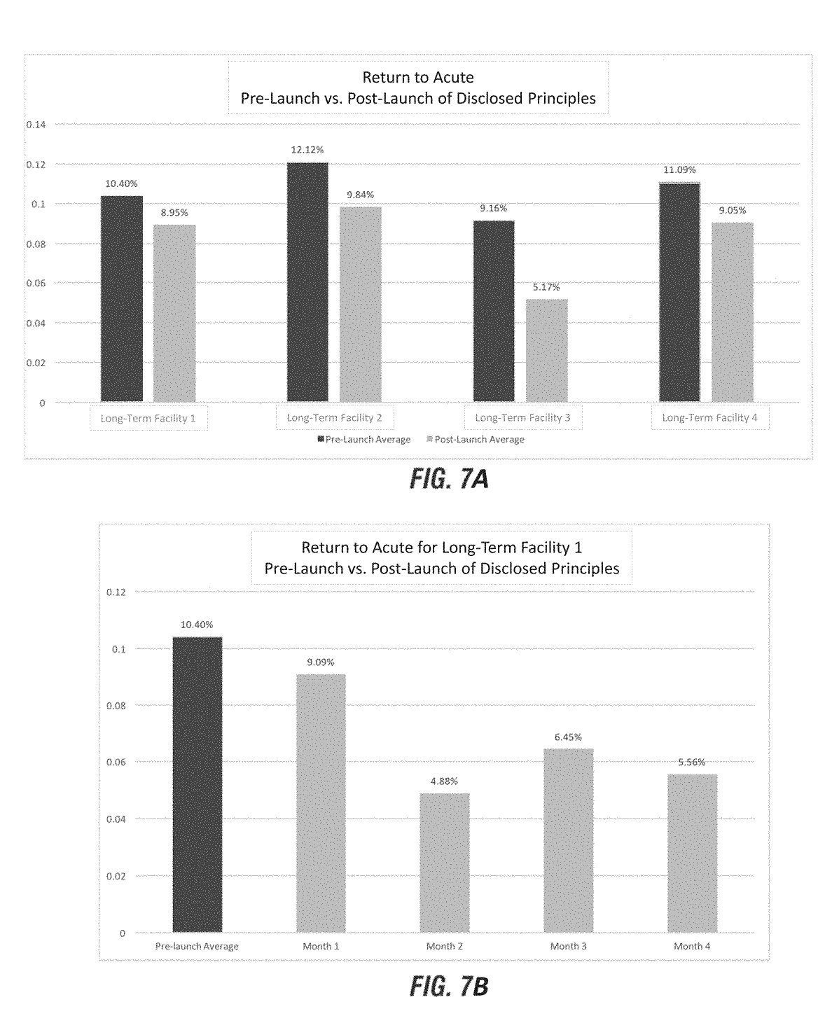

Looking finally at FIGS. 7A and 7B, illustrated are a graphs of actual average transfer rates both before and after implementing a telemedicine program in accordance with the disclosed principles. Generally speaking, such transfer rates, or “send-out” rates, can include RTAs, but also include any transfer of a patient from a lower level healthcare facility to a higher level (relative to the lower level facility) facility based on a condition of the patient being traditionally better suited for the higher level facility. However, as discussed in detail above, many such send-outs or transfers were not required, but the lack of higher level (i.e., higher specialty) physician access at the lower level facility still led to the patient being transferred nonetheless. The graph of FIG. 7A specifically illustrates the actual average RTA transfer rates determined by the present inventors of all of the patients in four distinct long-term health care (LTC) facilities to an acute care facility, averaged over a six month period.

The pre-launch average RTA transfer rates for each of the four LTC Facility facilities can be seen on the left side of the plot for each LTC facility on the graph, while the post-launch transfer rates from each LTC facility is on the right side of each plot. For the first LTC facility, the RTA transfer rate was averaging 10.40% of patients before implementation of a telemedicine program in accordance with the disclosed principles, and the average RTA transfer rate dropped to 8.95% after implementation of the disclosed principles. Similarly, the average RTA transfer rate from the second LTC facility was 12.12% before implementation of the disclosed principles, and the RTA transfer rate dropped to an average of 9.84% after implementation of the disclosed principles. The average RTA transfer rate from the third LTC facility was 9.16% before implementation of the disclosed principles, and the average RTA transfer rate from that facility dropped to just 5.17% after implementation of the disclosed principles. And for the fourth LTC facility, the average RTA transfer rate was 11.09% before implementation of the disclosed principles, and the average RTA transfer rate dropped to 9.05% after implementation of the disclosed principles.

Looking now at FIG. 7B, the graph here provides a more detailed view of the RTA transfer rates of patients from the first LTC facility illustrated in FIG. 7A. Specifically, FIG. 7B illustrates the reduction in RTA transfer rate for the first four months after a telemedicine system and process in accordance with the disclosed principles was implemented at LTC facility 1. As discussed above, the average RTA transfer rate from LTC facility 1 to an acute care facility when patients were experiencing symptoms indicative of the need for acute care was averaging about 10.40% before implementation of the disclosed principles. After the first month of implementation of the disclosed principles, RTA transfers dropped to 9.09% of all patients in LTC facility 1. Then after the second month, the RTA transfer rate from LTC facility 1 to an acute care facility dropped all the way down to 4.88% of patients. After the third month, the RTA transfer rate was 6.45% of patients in LTC facility 1, which while higher the RTA transfer rate of the second month, it was still far lower than the 10.40% of patients being transferred prior to implementation of a telemedicine program in accordance with the disclosed principles. And for the fourth month, the RTA transfer rate for LTC facility 1 was down again to 5.56% of patients.

These reductions in patient transfer rates from a long-term, non-acute care facility to an acute care facility directly, which are empirically shown to be provided by a telemedicine program in accordance with the disclosed principles, result in a reduction of costs to the patients, the facilities, insurance companies, and social healthcare programs, as detailed above, since transfer to an acute care facility typically includes the additional cost for the physical transfer of the patient, as well as the traditionally higher cost of care in an acute care facility as compared to the transferring long-term non-acute care facility. Moreover, for those transferred patients who are later determined by the acute care facility to not have needed transferring, there are even more costs associated with the transfer back to the non-acute, or in general the lower level, facility that unnecessarily transferred them to begin with. Also, as discussed above, a telemedicine program in accordance with the disclosed principles eliminates the need to keep one or more physician specialists staffed at the lower level facility, such as a long-term non-acute care facility. Still further, physical specialists remotely located from such non-acute care facilities are able to assist many different non-acute care facilities, rather than being limited to a single non-acute care or acute care facility where they are employed. And as also discussed above, this technological ability to remotely assist any number of non-acute care facilities has no geographical limit, and thus physician specialist diagnosis and treatment can be extended into those locations where such medical specialty is difficult to find or even completely non-existent.

In the numerous embodiments of the inventive subject matter disclosed herein, such embodiments may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus the following claims are hereby incorporated into the Detailed Description, with each claim standing on its own as a separate embodiment.

The description has made reference to several exemplary embodiments. It is understood, however, that the words that have been used are for description and illustration, rather than words of limitation. Changes may be made within the purview of the appended claims, as presently stated and as amended, without departing from the scope and spirit of the disclosure in all its aspects. Although this description makes reference to particular means, materials and embodiments, the disclosure is not intended to be limited to the particulars disclosed; rather, the disclosure extends to all functionally equivalent technologies, structures, methods and uses such as are within the scope of the appended claims.

What is claimed is:

1. A method for reducing Return To Acute Care transfers (RTAs) of patients in a non-acute care facility, the method comprising:
determining if a patient currently provided for in a non-acute care facility is suffering one or more newly arising symptoms that may require acute care;
deploying a Telemedicine Acute Care Unit for the patient, while the patient remains in the non-acute care facility, if the patient is determined to be suffering such new symptoms;
diagnosing the patient's newly arising symptoms by a physician specialist, geographically remote from the non-acute care facility, using the Telemedicine Acute Care Unit, wherein the physician specialist controls functions of the Telemedicine Acute Care Unit to diagnose the patient and communicate with the patient and non-physician specialist personnel located with the patient;
if the remote physician specialist determines that the diagnosis of the patient requires transfer to an acute care facility, instructing such transfer via the Telemedicine Acute Care Unit;
if the remote physician specialist determines that the diagnosis of the patient does not require transfer to an acute care facility, instructing a treatment plan for the patient via the Telemedicine Acute Care Unit and for implementation by non-physician specialist personnel at the non-acute care facility.

2. A method in accordance with claim 1, wherein the one or more newly arising symptoms that may require acute care are one or more symptoms associated with acute conditions selected from the group consisting of:
a heart attack;
hemodynamically unstable gastrointestinal bleeding;
hemodynamically unstable pulmonary embolism;
conditions requiring urgent surgical intervention;
a neurologic event requiring a neurologist evaluation; and
static epilepticus (continues seizures).

3. A method in accordance with claim 1, wherein deploying a Telemedicine Acute Care Unit for the patient for diagnosing the patient's newly arising symptoms by a physician specialist geographically remote from the non-acute care facility comprises deploying the Telemedicine Acute Care Unit by the patient.

4. A method in accordance with claim 1, wherein deploying a Telemedicine Acute Care Unit for the patient for diagnosing the patient's newly arising symptoms by a physician specialist geographically remote from the non-acute care facility comprises deploying the Telemedicine Acute Care Unit by the remote physician specialist based on a remote monitoring of the patient's vital signs signaling the one or more newly arising symptoms that may require acute care.

5. A method in accordance with claim 1, wherein diagnosing the patient's newly arising symptoms by the remote physician specialist comprises receiving data generated by monitoring healthcare equipment of the patient via data connections between the healthcare equipment and the Telemedicine Acute Care Unit.

6. A method in accordance with claim 5, wherein the diagnosing further comprises conducting one or more medical tests on the patient as instructed by the remote physician specialist via the Telemedicine Acute Care Unit.

7. A method in accordance with claim 1, wherein diagnosing the patient's newly arising symptoms by the remote physician specialist comprises receiving telemetry data generated by telemetry devices in contact with the patient and
connected to the Telemedicine Acute Care Unit, said telemetry data simulating said contact with the patient for the remote physician specialist.

8. A method in accordance with claim 1, wherein instructing a treatment plan for the patient via the Telemedicine Acute Care Unit and for implementation by non-physician specialist personnel at the non-acute care facility comprises transmitting treatment plan instructions or information simultaneously to multiple departments in the non-acute care facility via the Telemedicine Acute Care Unit.

9. A method in accordance with claim 1, further comprising:
determining, by the remote physician specialist via the Telemedicine Acute Care Unit, if the instructed treatment plan implemented by the non-physician specialist personnel at the non-acute care facility has successfully eliminated the one or more symptoms requiring acute care;
if the instructed treatment plan is determined to be successful in eliminating the one or more symptoms requiring acute care, instructing, by the remote physician specialist via the Telemedicine Acute Care Unit, the non-physician specialist personnel at the non-acute care facility to cease the treatment plan; and
if the instructed treatment plan is determined to not be successful in eliminating the one or more symptoms requiring acute care, re-diagnosing the patient, by the remote physician specialist via the Telemedicine Acute Care Unit, for one or more existing or newly arising symptoms that may require acute care, and:
if the remote physician specialist determines that the re-diagnosis of the patient requires transfer to an acute care facility, instructing such transfer via the Telemedicine Acute Care Unit;
if the remote physician specialist determines that the re-diagnosis of the patient does not require transfer to an acute care facility, instructing a new treatment plan for the patient via the Telemedicine Acute Care Unit and for implementation by non-physician specialist personnel at the non-acute care facility.

10. A method in accordance with claim 1, further comprising:
determining, by a new remote physician specialist via the Telemedicine Acute Care Unit, if the instructed treatment plan implemented by the non-physician specialist personnel at the non-acute care facility has successfully eliminated the one or more symptoms requiring acute care;
if the instructed treatment plan is determined to be successful in eliminating the one or more symptoms requiring acute care, instructing, by the new remote physician specialist via the Telemedicine Acute Care Unit, non-physician specialist personnel at the non-acute care facility to cease the treatment plan; and
if the instructed treatment plan is determined to not be successful in eliminating the one or more symptoms requiring acute care, re-diagnosing the patient, by the new remote physician specialist via the Telemedicine Acute Care Unit, for one or more existing or newly arising symptoms that may require acute care, and:
if the new remote physician specialist determines that the re-diagnosis of the patient requires transfer to an acute care facility, instructing such transfer via the Telemedicine Acute Care Unit;
if the new remote physician specialist determines that the re-diagnosis of the patient does not require transfer to an acute care facility, instructing a new treatment plan for the patient via the Telemedicine Acute Care Unit and for implementation by non-physician specialist personnel at the non-acute care facility.

11. A telemedicine unit for providing remote diagnosing and treating for patients by geographically remote physicians, the telemedicine unit comprising:
a support structure configured to secure components of telemedicine unit in a single moveable unit, the support structure comprising a base and a mast vertically extending therefrom;
a display unit connected to the support structure and configured to present live video of a remotely located physician employing the telemedicine unit to diagnose and treat a patient from a geographically remote location;
one or more audio speakers associated with the display unit and configured to provide audio from the remote physician to the patient's location during the remote diagnosis or treating of the patient by the remote physician;
a moveable camera unit connected to the support structure and configured to be remotely controlled by the remote physician using one or more of pan, tilt, zoom and focus commands to the camera unit;
a microphone associated with the camera unit and configured to capture audio from the patient's location and side and provide it to the remote physician during the remote diagnosis or treating of the patient by the remote physician;
a communications unit connected to the support structure and configured to be connected to a communications network at the patient's location for transmitting and receiving data regarding the patient with communications equipment employed by the remote physician during the remote diagnosis or treating of the patient by the remote physician, the communications unit comprising one or more peripheral input devices for providing information to the communications unit during the remote diagnosis or treating of the patient by the remote physician;
a power supply connected to the support structure and configured to provide electrical power to components of the telemedicine unit via wired electrical connection to a standard electrical outlet and via battery power when wired electrical power is not present; and
a plurality of wheels connected to the base and configured to provide mobility of movement to the telemedicine unit, at least one of the plurality of wheels comprising a wheel lock for preventing movement of the telemedicine unit when engaged.

12. A telemedicine unit in accordance with claim 11, wherein the mast of the support structure is hollow and configured to receive cabling associated with components of the telemedicine unit therein.

13. A telemedicine unit in accordance with claim 11, wherein the display unit is sized such that the remote physician is displayed thereon life-size.

14. A telemedicine unit in accordance with claim 11, wherein the telemedicine unit is configured to be accessed by remote physicians through video conferencing hardware and software executed on a desktop computer, a mobile communications device application, an internet browser, or dedicated video conferencing equipment.

15. A telemedicine unit in accordance with claim 11, wherein the display unit is further configured to display images or videos pertaining to the diagnosis or treatment of the patient provided by the remote physician.

16. A telemedicine unit in accordance with claim 11, further comprising a self-diagnostics unit configured to monitor status and performance of the telemedicine unit, and including automated alerts triggered when one or more components of the telemedicine unit is determined to be functioning below a predetermined threshold.

17. A telemedicine unit in accordance with claim 11, wherein the communications unit is configured to communicate via each of a wireless local area network, a wired local area network, and a wireless cellular network.

18. A telemedicine unit in accordance with claim 11, wherein the communications unit is further configured with prioritization capability configured to take priority of resources of a network to which it is connected over other devices connected to the network.

19. A telemedicine unit in accordance with claim 18, wherein the communications unit is configured to take priority of resources of the network based on a determination of available bandwidth on the network upon connection to the network.

20. A telemedicine unit in accordance with claim 18, wherein the communications unit is configured to take priority of resources of the network based on a type of medical procedure being performed on the patient by the remote physician with the telemedicine unit.

21. A telemedicine unit in accordance with claim 11, further comprising a telemetry input unit configured to receive information from equipment in physical contact with the patient and provide that information to the communications unit, the communications unit further configured to provide that information to the remote physician during the remote diagnosis or treating of the patient by the remote physician.

22. A telemedicine unit in accordance with claim 21, wherein the equipment comprises tactile-based equipment selected from the group consisting of:
a stethoscope,
an otoscope,
an ophthalmoscope, and
telemetry gloves configured to mimic tactile sensations experienced from a wearer touching the patient to the remote physician.

23. A telemedicine unit accordance with claim 11, further comprising a backup conferencing unit connected to the communications unit, wherein the backup conferencing unit includes video capture and display capabilities and audio capture and broadcast capabilities, wherein the backup conference unit can be activated if one or more of the display unit, camera, microphone or speakers ceases functioning, and wherein the backup conferencing unit's video capture and display capabilities and audio capture and broadcast capabilities thereby function in place of the display unit, camera, microphone and speakers.
